(19) United States
(12) Patent Application Publication
(10) Pub. No.: US 2025/0032072 A1
(43) Pub. Date: Jan. 30, 2025

(54) INFORMATION PROCESSING DEVICE, INFORMATION PROCESSING METHOD, AND PROGRAM

(71) Applicant: CANON KABUSHIKI KAISHA, Tokyo (JP)

(72) Inventor: DAISUKE YAMADA, Kanagawa (JP)

(21) Appl. No.: 18/778,253

(22) Filed: Jul. 19, 2024

(30) Foreign Application Priority Data
Jul. 26, 2023 (JP) 2023-121792

(51) Int. Cl. A61B 6/46; A61B 6/00; A61B 6/42; G01T 1/24; G06T 3/40; G06T 5/60; G06T 5/70; G06T 7/00

(52) U.S. Cl. CPC A61B 6/463; A61B 6/42; A61B 6/5258; G01T 1/24; G06T 3/40; G06T 5/60; G06T 5/70; G06T 7/0012; G06T 2207/20081; G06T 2207/30004

(57) ABSTRACT

An information processing device according to an embodiment of the present disclosure includes one or more memories storing instructions and one or more processors that, upon execution of the stored instructions, are configured to operate as: a display control unit configured to, upon receiving an instruction for acquiring a first radiation image, control a display unit such that a second radiation image is displayed first and then the first radiation image is displayed. The first radiation image is obtained by performing first image processing upon a radiation image obtained by detecting a radiation. The second radiation image is obtained by performing second image processing requiring a shorter processing time than the first image processing upon the radiation image.

### BACKGROUND

### Field

The present disclosure relates to an information processing device, an information processing method, and a program.

### Description of the Related Art

Radiation imaging devices each using a planar detector (flat panel detector (FPD)) made of a semiconductor material are widely used in medical image diagnoses or nondestructive inspections. Image processing is performed upon a radiation image obtained by using the FPD for the purpose of, for example, noise reduction.

Japanese Patent Laid-Open No. 2023-57871 discloses a device for performing image processing using machine learning for the purpose of noise reduction.

Image processing using machine learning may take a longer processing time than, for example, image processing not using machine learning (e.g., image processing using a smoothing filter). As a result, for example, a period from a time at which a radiographer performs imaging to a time at which a radiation image is displayed on a display unit may become longer. This may lead to the increase in a time required for a diagnosis.

### SUMMARY

The present disclosure shortens the time it takes to display a radiation image on a display unit when image processing that takes a long processing time is performed.

An information processing device according to an embodiment of the present disclosure includes one or more memories storing instructions and one or more processors that, upon execution of the stored instructions, are configured to operate as: a display control unit configured to, upon receiving an instruction for acquiring a first radiation image, control a display unit such that a second radiation image is displayed first and then the first radiation image is displayed. The first radiation image is obtained by performing first image processing upon a radiation image obtained by detecting a radiation. The second radiation image is obtained by performing second image processing requiring a shorter processing time than the first image processing upon the radiation image.

Further features of the present disclosure will become apparent from the following description of exemplary embodiments with reference to the attached drawings.

### BRIEF DESCRIPTION OF THE DRAWINGS

FIG. 1 is a diagram illustrating an exemplary configuration of a radiation imaging system according to a first embodiment.

FIG. 2 is a flowchart illustrating the operation of a controller according to the first embodiment.

FIGS. 3A and 3B are flowcharts illustrating details of the operation of the controller according to the first embodiment.

FIG. 4 is a flowchart illustrating the operation of a controller according to a second embodiment.

FIG. 5 is a diagram illustrating an exemplary configuration of a radiation imaging system according to a third embodiment.

FIGS. 6A to 6C are diagrams illustrating image displays according to the third embodiment.

FIG. 7 is a diagram illustrating an exemplary configuration of a radiation imaging system according to a fourth embodiment.

FIG. 8 is a flowchart illustrating the operation of a controller according to the fourth embodiment.

FIGS. 9A and 9B are diagrams illustrating image displays according to the fourth embodiment.

### DESCRIPTION OF THE EMBODIMENTS

Embodiments of the present disclosure will be described below with reference to the accompanying drawings. The embodiments to be described below do not limit the present disclosure according to the claims. All of combinations of the features described in the embodiments are not always essential to the means for solution according to the present disclosure.

### First Embodiment

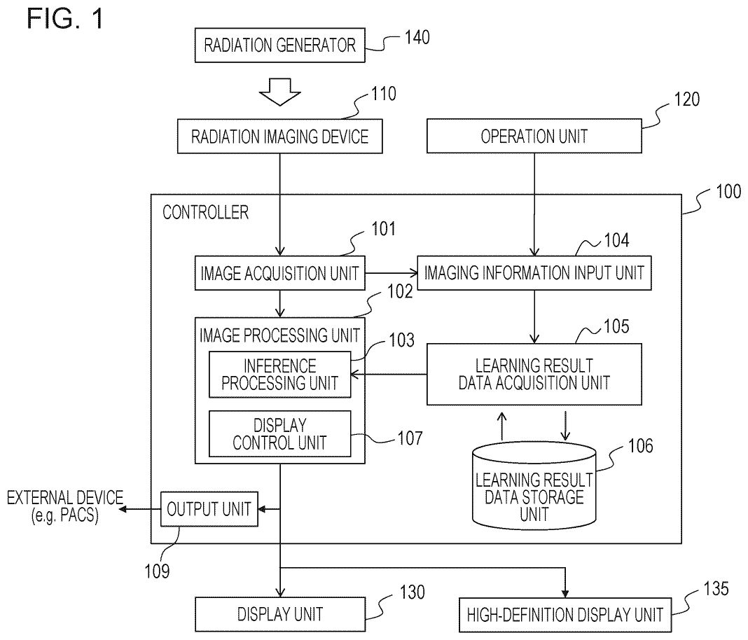

FIG. 1 is a diagram illustrating an exemplary configuration of a radiation imaging system according to a first embodiment. A radiation imaging system includes a controller 100, a radiation imaging device 110, an operation unit 120, a display unit 130, a high-definition display unit 135, and a radiation generator 140.

The radiation imaging device 110 is, for example, a radiation imaging device that uses a planar detector (flat panel detector (FPD)) made of a semiconductor material. The radiation imaging device 110 detects a radiation that has passed through a subject (not illustrated) and outputs image data corresponding to the radiation. Image data can also be referred to as a medical image or a radiation image. The radiation imaging device 110 converts the radiation that has passed through the subject into an electric charge.

For example, a direct conversion type sensor for directly converting a radiation into an electric charge using, for example, amorphous selenium (a-Se) is used for the conversion into an electric charge. For example, an indirect type sensor for converting a radiation into visible light and then converting the visible light into an electric charge using a scintillator, such as cesium iodide (CsI), and a photoelectric conversion element, such as amorphous silicon (a-Si), is used for the conversion into an electric charge. The radiation imaging device 110 generates image data by performing analog-to-digital (A/D) conversion upon an electric charge. The radiation imaging device 110 outputs the generated image data to the controller 100.

The operation unit 120 is, for example, a mouse or a keyboard for receiving an instruction from an operator.

The display unit 130 is, for example, a liquid crystal display. The display unit 130 is controlled by a display control unit 107 included in the controller 100. The display unit 130 may be a separate body from the controller 100 or may be incorporated into the controller 100.

The high-definition display unit 135 is, for example, a liquid crystal display that has a higher resolution than the display unit 130. The high-definition display unit 135 is controlled by the display control unit 107 included in the controller 100. The high-definition display unit 135 may be a separate body from the controller 100 or may be incorporated into the controller 100.

The display unit 130 and the high-definition display unit 135 do not necessarily have to be separate units. For example, the high-definition display unit 135 may have two display regions.

The controller 100 is, for example, an information processing device, such as a computer. For example, the controller 100 is connected to the radiation imaging device 110 in a communicable manner via a wired or wireless network. The communicable manner includes not only direct communication between devices but also communication between the devices via another device. The controller 100 can execute an application. That is, the controller 100 includes one or more processors and memories and realizes each functional unit to be described below by causing the processor to execute a program stored in the memory. A part or all of each functional unit may be realized by dedicated hardware. The controller 100 performs image processing upon image data input from the radiation imaging device 110. The controller 100 displays the image data that has been subjected to the image processing on the display unit 130 or the high-definition display unit 135. The display of the image data on the display unit 130 or the high-definition display unit 135 is performed by the display control unit 107 included in the controller 100. The display control unit 107 is, for example, a display driver incorporated into a CPU. The display control unit 107 is an example of a display control unit.

The controller 100 displays a graphical user interface (GUI) on the display unit 130. The controller 100 controls a timing at which the radiation generator 140 generates a radiation and radiation imaging conditions.

The controller 100 includes an image acquisition unit 101, an image processing unit 102, an imaging information input unit 104, a learning result data acquisition unit 105, and a learning result data storage unit 106, and an output unit 109.

The image acquisition unit 101 acquires image data generated by the radiation imaging device 110.

The imaging information input unit 104 is, for example, a GUI. Imaging information is input (set) to the imaging information input unit 104 by an operator. The imaging information includes, for example, the type of a radiation imaging device, the type of an image to be processed, an image processing method, or an image display method. For example, the type of a radiation imaging device includes, for example, a fluorescent material, the imaging resolution of a radiation imaging device, and the pixel pitch of the radiation imaging device. The type of an image to be processed includes, for example, a ⅛ thinned-out preview image, a ¼ thinned-out preview image, a ½ thinned-out preview image, a full size image without thinning out, and an output image based on the size and preprocessing of an image. The image processing method includes, for example, image processing using machine learning and image processing not using machine learning. The image display method includes, for example, a display method of displaying only a radiation image that has been subjected to the image processing using machine learning and a display method of displaying a radiation image that has been subjected to the image processing not using machine learning before displaying a radiation image that has been subjected to the image processing using machine learning. An operator inputs imaging information to the GUI using the operation unit 120. Imaging information acquired by the image acquisition unit 101 is input to the imaging information input unit 104. The imaging information input to the imaging information input unit 104 is associated with image data acquired by the image acquisition unit 101.

The learning result data storage unit 106 stores learning result data obtained by machine learning using a teacher image.

The learning result data acquisition unit 105 reads out the learning result data from the learning result data storage unit 106. The learning result data acquisition unit 105 stores information that associates various words and a combination of words, which are included in the imaging information, with the learning result data stored in the learning result data storage unit 106. Accordingly, the learning result data acquisition unit 105 can acquire the learning result data to be used for processing of an inference processing unit 103 included in the image processing unit 102 based on the word included in the imaging information.

The image processing unit 102 includes the inference processing unit 103 and the display control unit 107.

The inference processing unit 103 performs inference processing using the learning result data obtained by machine learning, such as noise reduction processing. The inference processing unit 103 may perform inference processing other than the noise reduction processing, such as gradation processing.

In addition to the inference processing, the image processing unit 102 can also perform image processing, such as contrast adjustment, upon image data input from the radiation imaging device 110. The image processing unit 102 can also perform image processing, such as trimming and rotation, upon the image data input from the radiation imaging device 110. The image processing unit 102 can perform not only the image processing using machine learning (the inference processing of the inference processing unit 103) but also the image processing not using machine learning (e.g., image processing using a smoothing filter). Based on the resolution of a connected display unit or the imaging information input to the imaging information input unit 104, it is determined which of these pieces of image processing is to be performed. A processing time required for the image processing using machine learning is longer than a processing time required for the image processing not using machine learning. The image processing using machine learning is an example of first image processing. The image processing not using machine learning is an example of second image processing.

The display control unit 107 controls the display unit 130 and the high-definition display unit 135. The display control unit 107 can detect whether a display unit is connected thereto. The detection is performed based on, for example, display setting information set by an operator. The display setting information is, for example, information indicating the number of display units (or display regions) connected to the controller 100.

The display setting information is, for example, information indicating the arrangement relationship between a plurality of display units (or display regions). The detection is performed by, for example, the electric communication between the display unit and the controller 100. The electric communication includes, for example, a connection detection signal transmitted from the display unit to the controller 100 when the display unit and the controller 100 are connected to each other by a cable. The electric communication also includes, for example, communication for a negotiation performed after the connection detection signal has been transmitted. The display control unit 107 can acquire the resolution of the display unit connected thereto.

The resolution is acquired from the above-described display setting information or by the above-described electric communication. Accordingly, the display control unit 107 can detect whether the display unit 130 and the high-definition display unit 135 are connected thereto. The display unit 130 and the high-definition display unit 135 are examples of a display unit. The display unit 130 is an example of a first display region. The high-definition display unit 135 is an example of a second display region different from the first display region. A GUI used for the setting of imaging conditions is displayed on any one of the display unit 130 and the high-definition display unit 135 and is not displayed on the other one of them. In this case, it is assumed that a GUI is displayed on the display unit 130.

The output unit 109 outputs an image that has been subjected to image processing by the image processing unit 102 to an external device (e.g., a picture archiving and communication system (PACS) or a printer).

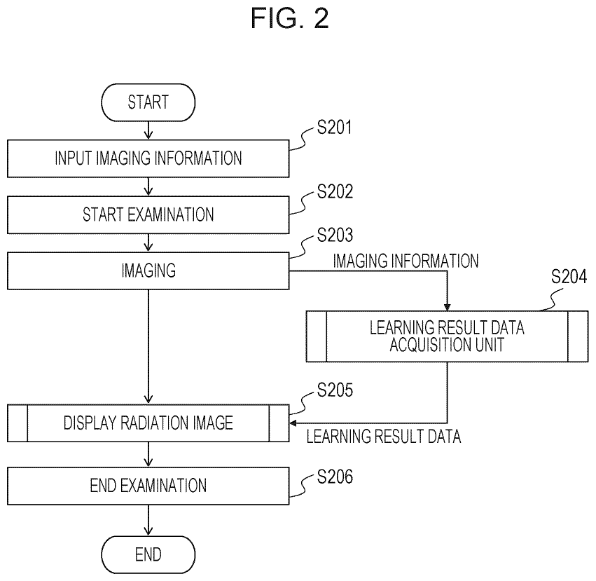

Next, the operation of the controller 100 according to the first embodiment will be described with reference to the flowchart illustrated in FIG. 2.

In step S201, imaging information is input to the imaging information input unit 104 included in the controller 100. For example, the imaging information is input in such a manner that an operator selects one of a plurality of pieces of imaging information displayed on the display unit 130 in, for example, list form.

The imaging information does not necessarily have to be selected by an operator and may be directly input by an operator. These inputs performed by an operator are examples of an instruction for acquisition. In this case, it is assumed that the acquisition of a radiation image that has been subjected to the image processing using machine learning is instructed. The instruction for acquisition may be, for example, an examination start instruction. Alternatively, the instruction for acquisition may be, for example, an instruction indicating that an examination is ready to be started. That is, the instruction for acquisition may be any instruction input to the controller 100 in the process of imaging.

In step S202, the controller 100 starts an examination based on the imaging information input in step S201. Specifically, the controller 100 transmits an imaging preparation start signal to the radiation imaging device 110. Upon receiving the imaging preparation start signal, the radiation imaging device 110 controls a bias power supply using a main control circuit and applies a bias voltage to imaging pixels. Subsequently, the radiation imaging device 110 performs an initialization operation of reading out electric charges from the imaging pixels using a driving circuit to read out a dark current signal stored in the imaging pixels. After the completion of the initialization operation, the radiation imaging device 110 transmits state information indicating that an imaging preparation has been done to the controller 100. The controller 100 sets an operation parameter (e.g., a tube voltage) of the radiation generator 140 based on the imaging information selected in step S201. Upon receiving the state information from the radiation imaging device 110, the controller 100 notifies the radiation generator 140 of an exposure permission.

In step S203, the image acquisition unit 101 included in the controller 100 acquires a radiation image captured by the radiation imaging device 110. Specifically, for example, the radiation generator 140 that has been notified of the exposure permission emits a radiation in response to the operation of an exposure button. The driving circuit of the radiation imaging device 110 reads out an acquired image signal obtained by the detection of an emitted radiation using a readout circuit and generates a radiation image. Subsequently, the radiation imaging device 110 transmits the generated radiation image to the controller 100. The image acquisition unit 101 included in the controller 100 receives the radiation image transmitted by the radiation imaging device 110. In the process from step S201 to step S203, the controller 100 associates the imaging information input to the imaging information input unit 104 and the radiation image (image data) acquired by the image acquisition unit 101 with each other.

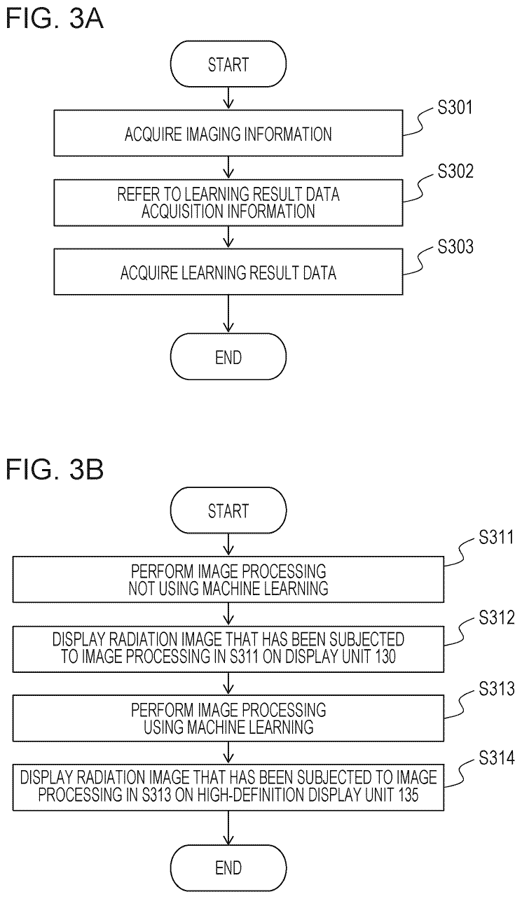

In step S204, the learning result data acquisition unit 105 included in the controller 100 acquires learning result data from the learning result data storage unit 106 based on the imaging information selected in step S203. The processing of step S204 will be described in detail below with reference to the flowchart illustrated in FIG. 3A. The processing of step S204 is performed in parallel with the processing of step S203 (the acquisition of a radiation image).

In step S205, the image processing unit 102 included in the controller 100 performs image processing upon the radiation image captured by the radiation imaging device 110. The display control unit 107 included in the controller 100 displays the radiation image that has been subjected to the image processing on a display unit. The processing of step S205 is a feature part of the present embodiment, and will therefore be described in detail below with reference to the flowchart illustrated in FIG. 3B.

After the radiation image has been displayed on the display unit, the controller 100 ends the examination in response to the input operation of the operator in step S206.

Next, learning result data acquisition processing (the processing of step S204) performed by the learning result data acquisition unit 105 included in the controller 100 will be described with reference to the flowchart illustrated in FIG. 3A.

In step S301, the learning result data acquisition unit 105 acquires the imaging information determined in step S203.

In step S302, the learning result data acquisition unit 105 selects learning result data based on the imaging information acquired in step S301.

In step S303, the learning result data acquisition unit 105 reads out the learning result data selected in step S302 from the learning result data storage unit 106, decompresses the learning result data so that the inference processing unit 103 can use it, for example, and stores the learning result data in a storage unit (not illustrated).

Next, the processing of the image processing unit 102 and the display control unit 107 included in the controller 100 (the processing of step S205) will be described with reference to the flowchart illustrated in FIG. 3B.

In step S311, the image processing unit 102 performs the image processing not using machine learning.

Subsequently, in step S312, the display control unit 107 displays a radiation image that has been subjected to the image processing in S311 on the display unit 130. The radiation image that has been subjected to the image processing in S311 is an example of a second radiation image.

Subsequently, in step S313, the image processing unit 102 performs the image processing using machine learning using the learning result data acquired by the processing of S204.

Subsequently, in step S314, the display control unit 107 displays a radiation image that has been subjected to the image processing in S313 on the high-definition display unit 135. The radiation image that has been subjected to the image processing in S313 is an example of a first radiation image.

In a method in the related art, the image processing not using machine learning and the display (S311 to S312) are not performed and the image processing using machine learning and the display (S313 to S314) are performed. That is, a processing time required for the image processing using machine learning is needed before a captured radiation image is displayed on the display unit.

In the method according to the present embodiment, the image processing not using machine learning and the display (S311 to S312) are performed before the image processing using machine learning and the display (S313 to S314). A processing time required for the image processing not using machine learning is shorter than a processing time required for the image processing using machine learning. Accordingly, with the method according to the present embodiment, the time it takes to display a captured radiation image on a display unit can be shortened as compared with the case where the method in the related art is employed.

In the case where the controller 100 can perform the multiple pieces of image processing not using machine learning, the controller 100 may select one of them depending on the purpose. For example, the controller 100 may select the image processing with the shortest processing time when giving priority to a time taken to display. For example, the controller 100 may select the image processing in which a processing result most similar to a processing result of the image processing using machine learning is obtained. In this case, not only can a time taken to display be shortened, but a radiographer can appropriately check an imaging result. The selection may be performed by the controller 100 or an operator, such as a radiographer.

The radiation image displayed on the display unit 130 is used for, for example, the check of a radiation image imaging result by a radiographer or the adjustment of an image processing parameter. Accordingly, the operational efficiency of a radiographer can be improved by shortening the time it takes to display a captured radiation image on the display unit 130.

The check of a radiation image to be output to an external device is performed using the high-definition display unit 135 after a radiographer has performed a check and adjustment. Accordingly, the time it takes to output the radiation image to the external device can be shortened by shortening the time it takes to display a captured radiation image on the display unit 130.

A doctor makes a diagnosis after a radiographer has performed a check and adjustment. Accordingly, the diagnosis can be made quickly by shortening the time it takes to display a captured radiation image on the display unit 130.

### Second Embodiment

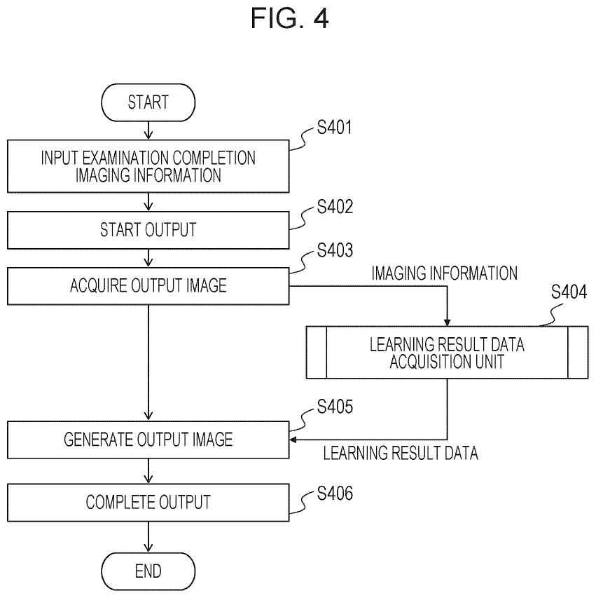

The operation of the controller 100 from imaging to display has been described in the first embodiment. A medical image, such as a captured radiation image, is generally output to an external device for an image check, which is connected to an in-hospital network, and is used for, for example, a diagnosis. In the second embodiment, the operation of the controller 100 when a medical image captured in the first embodiment is output to an external device, such as a PACS or a printer, will be described. The operation of the controller 100 according to the second embodiment will be described with reference to the flowchart illustrated in FIG. 4.

In step S401, examination completion imaging information is input to the imaging information input unit 104 included in the controller 100. The examination completion imaging information includes, for example, the imaging date and time of a captured radiation image, a patient ID, and a patient name. The examination completion imaging information is input by, for example, causing an operator to select one of the multiple pieces of examination completion imaging information displayed on the display unit 130 in list form. The selected examination completion imaging information is set as a target to be output to an external device. The examination completion imaging information does not necessarily have to be selected by an operator and may be directly input by an operator.

In step S402, the controller 100 starts output processing based on the examination completion imaging information input in step S401.

In step S403, the image acquisition unit 101 included in the controller 100 acquires from an image storage unit (not illustrated) the radiation image associated with the examination completion imaging information.

In step S404, the learning result data acquisition unit 105 included in the controller 100 acquires the learning result data from the learning result data storage unit 106 based on the imaging information associated with the radiation image acquired in step S403.

In step S405, the image processing unit 102 included in the controller 100 performs image processing upon the radiation image acquired by the image acquisition unit 101. At that time, the inference processing unit 103 performs the inference processing by machine learning using the learning result data obtained in step S404. The inference processing unit 103 performs, for example, noise reduction processing and processing for determining contents of display, such as an annotation, to be superimposed on an output image. In S314 (display) in the first embodiment, the case has been described where the radiation image that has been subjected to the image processing using machine learning is displayed on the high-definition display unit 135. However, in the case of an output to, for example, a PACS, an image check is performed on the screen of the PACS that is an output destination. That is, the image processing in step S405 may differ from the image processing according to the first embodiment because an application differs from the application in the first embodiment. For example, the learning result data that prioritizes speed is used in the first embodiment, but the learning result data may be used differently in such a manner that the learning result data that prioritizes image quality is used in the image processing in step S405.

In step S406, the controller 100 ends the output.

As described above, according to the second embodiment, noise reduction processing using optimum learning result data suitable for output processing can also be performed in the output processing. As a result, an operator can check a radiation image of image quality equal to the image quality of a radiation image output to, for example, a PACS using the high-definition display unit 135.

### Third Embodiment

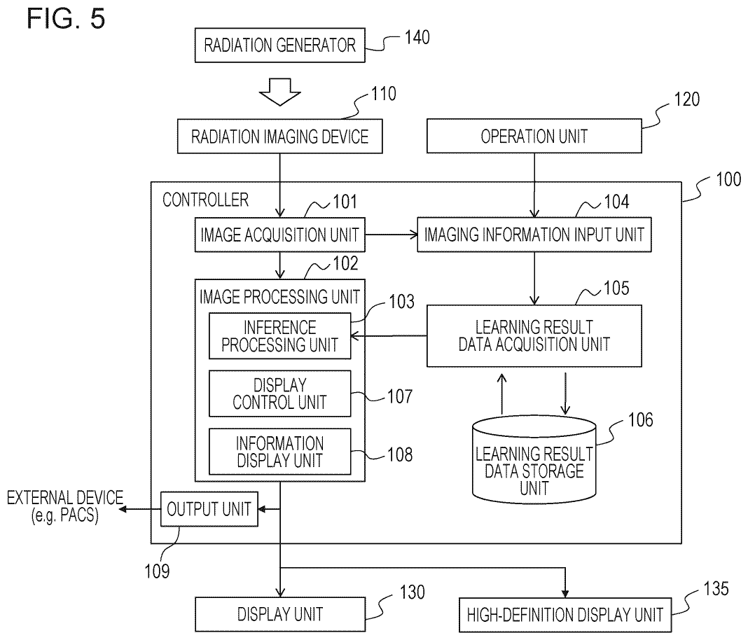

In the first embodiment, the image processing performed upon radiation images displayed on the display unit 130 and the high-definition display unit 135 has been described. FIG. 5 is a block diagram illustrating an exemplary configuration of a radiation imaging system according to a third embodiment. In FIG. 5, components similar to those according to the first embodiment are denoted by the same reference numerals.

A radiation imaging system according to the third embodiment further includes an information display unit 108 for displaying pieces of image processing information (image processing parameters) applied to respective radiation images to be displayed on the display unit 130 and the high-definition display unit 135.

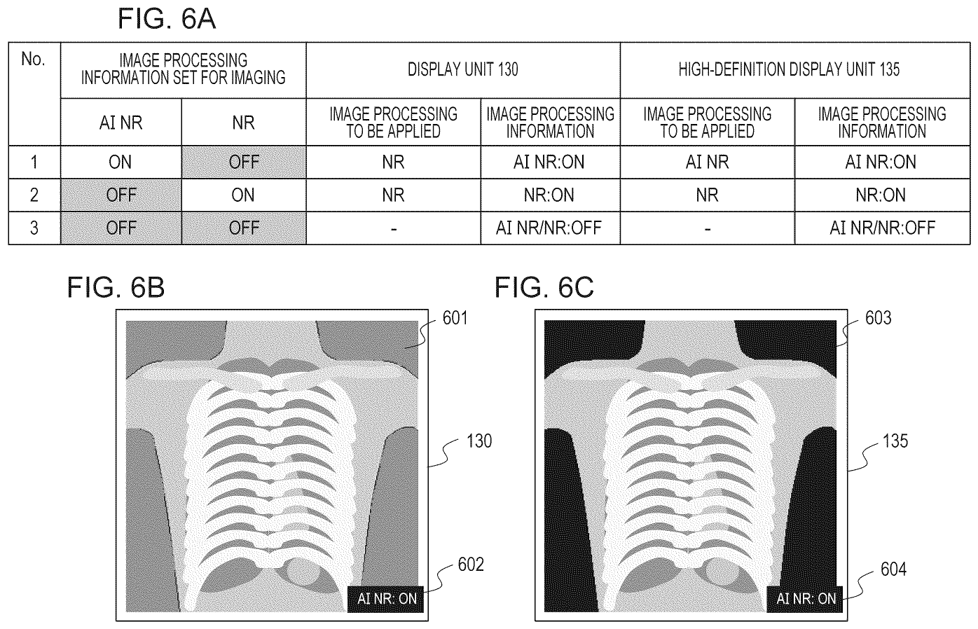

The information display unit 108 will be described with reference to FIGS. 6A to 6C. The table illustrated in FIG. 6A represents pieces of image processing information set for imaging, pieces of image processing applied to radiation images to be displayed on the display unit 130 and the high-definition display unit 135, and pieces of image processing information displayed by the information display unit 108. The image processing information set for imaging is information instructed by, for example, an operator. For example, No. 1 indicates that noise reduction processing using machine learning (AI NR) has been instructed to be performed. For example, No. 2 indicates that noise reduction processing not using machine learning (NR) has been instructed to be performed. For example, No. 3 indicates that neither the noise reduction processing using machine learning (AI NR) nor the noise reduction processing not using machine learning (NR) has been instructed to be performed.

FIGS. 6B and 6C illustrate exemplary displays of the display unit 130 and the high-definition display unit 135 in the case of No. 1 illustrated in FIG. 6A. FIG. 6B illustrates the exemplary display of the display unit 130. FIG. 6C illustrates the exemplary display of the high-definition display unit 135.

Referring to FIG. 6B, a radiation image 601 to which the noise reduction processing not using machine learning (NR) is applied is displayed on the display unit 130. On the radiation image 601, the information display unit 108 displays image processing information 602 (AI NR: ON) indicating that the noise reduction processing using machine learning (AI NR) is to be applied. The image processing information 602 is an example of the first image processing information.

Referring to FIG. 6C, the radiation image 603 corresponding to the image processing information set for imaging, to which the noise reduction processing using machine learning (AI NR) is applied, is displayed on the high-definition display unit 135. On the radiation image 603, the information display unit 108 displays image processing information 604 (AI NR: ON) indicating that the noise reduction processing using machine learning (AI NR) is to be applied.

As described above, with a method according to the third embodiment, the image processing and the display not using machine learning are performed upon the display unit 130 like in the first embodiment. With the method according to the third embodiment, the image processing and the display using machine learning are performed upon the high-definition display unit 135 like in the first embodiment. Accordingly, with the method according to the third embodiment, effects such as the improvement of operational efficiency of a radiographer can be achieved like in the first embodiment.

Furthermore, with the method according to the third embodiment, the image processing information 604 indicating that the image processing using machine learning (AI NR) is to be applied to the radiation image displayed on the display unit 130 which has been subjected to the image processing not using machine learning is displayed. Accordingly, an effect of allowing an operator to check image processing information to be applied to a radiation image to be output to, for example, a PACS can be achieved.

### Fourth Embodiment

In a fourth embodiment, the case will be described where the high-definition display unit 135 is not connected to the controller 100.

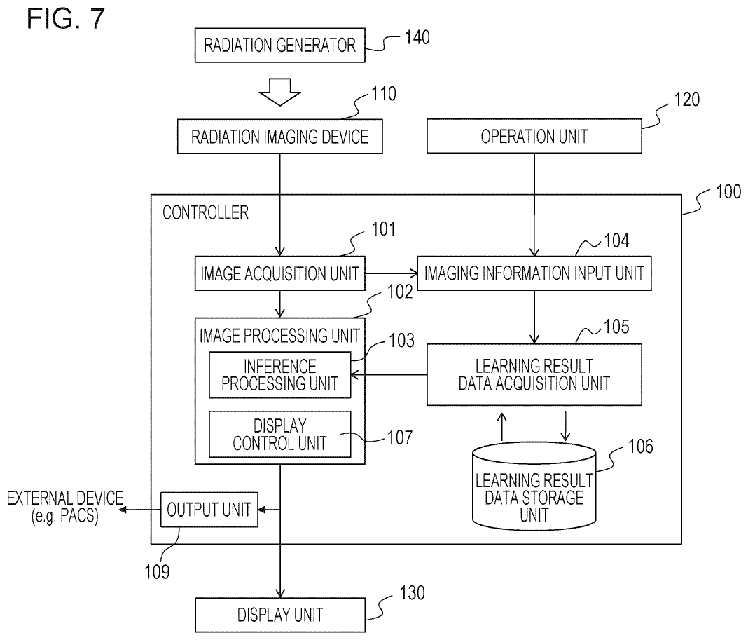

FIG. 7 is a block diagram illustrating an exemplary configuration of a radiation imaging system according to the fourth embodiment. The difference from FIG. 1 used in the description of the first embodiment is that the high-definition display unit 135 is not connected.

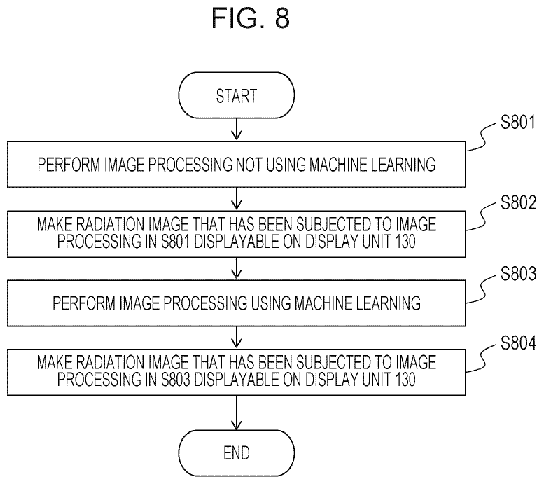

FIG. 8 is a flowchart describing the difference in the operation of the controller 100 according to the fourth embodiment from the first embodiment. The operation of the controller 100 according to the fourth embodiment differs from the operation of the controller 100 according to the first embodiment in the processing of S205 illustrated in FIG. 2 which has been used for the description of the first embodiment. Accordingly, FIG. 8 illustrates details of the processing of S205.

In step S801, the image processing unit 102 performs the image processing not using machine learning.

Subsequently, in step S802, the display control unit 107 performs control to make the radiation image that has been subjected to the image processing in S801 displayable on the display unit 130. For example, the control for making the radiation image displayable is performed such that the radiation image can be displayed in response to the operation (e.g., mouse click) of an operator. The control for making the radiation image displayable does not necessarily have to be performed, and the radiation image may be displayed on the display unit 130.

Subsequently, in step S803, the image processing unit 102 performs the image processing using machine learning using the learning result data acquired by the processing of S204.

Subsequently, in step S804, the display control unit 107 performs control to make the radiation image that has been subjected to the image processing in S803 displayable on the display unit 130.

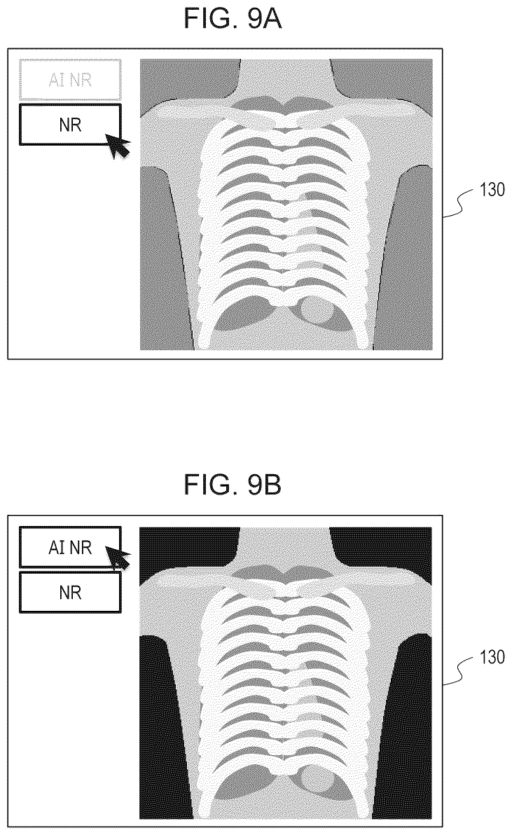

FIGS. 9A and 9B illustrate display examples. FIG. 9A illustrates the display state where an operator has clicked a button indicating NR (the image processing not using machine learning) on a GUI after the completion of step S802. In the state where step S802 has completed, the radiation image that has been subjected to the image processing not using machine learning is displayable. Accordingly, the radiation image that has been subjected to the image processing not using machine learning is displayed in response to a mouse click. On the other hand, in the state where step S802 has completed, a radiation image that has been subjected to the image processing using machine learning is not displayable. Accordingly, a display (gray display) indicates that selection is not available.

FIG. 9B illustrates the display state where the operator has clicked a button indicating AI NR (the image processing using machine learning) on the GUI after the completion of step S804. In the state where step S804 has completed, the radiation image that has been subjected to the image processing using machine learning is displayable. Accordingly, the radiation image that has been subjected to the image processing using machine learning is displayed in response to a mouse click.

As described above, with the method according to the fourth embodiment, the display of a radiation image that has been subjected to the image processing using machine learning and the display of a radiation image that has been subjected to the image processing not using machine learning can be switched even in the case where the high-definition display unit 135 is not connected.

Although the method of switching between the display of a radiation image and the display of a radiation image that has been subjected to the image processing not using machine learning has been described in the fourth embodiment, a display method is not limited thereto. For example, a radiation image and a radiation image that has been subjected to the image processing not using machine learning may be displayed side by side.

In the method according to the fourth embodiment, the image processing and the display not using machine learning (S801 to S802) are performed prior to the image processing and the display using machine learning (S803 to S804) like in the method according to the first embodiment. A processing time required for the image processing not using machine learning is shorter than a processing time required for the image processing using machine learning. Accordingly, with the method according to the present embodiment, the time it takes to display a captured radiation image on a display unit can be shortened as compared with the case where the method in the related art is employed.

According to the present disclosure, the time it takes to display a radiation image on a display unit can be shortened when image processing that takes a long processing time is performed.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2023-121792 filed Jul. 26, 2023, which is hereby incorporated by reference herein in its entirety.

What is claimed is:

1. An information processing device comprising:
one or more memories storing instructions; and
one or more processors that, upon execution of the stored instructions, are configured to operate as: a display control unit configured to, upon receiving an instruction for acquiring a first radiation image, control a display unit such that a second radiation image is displayed first and then the first radiation image is displayed, the first radiation image being obtained by performing first image processing upon a radiation image obtained by detecting a radiation, the second radiation image being obtained by performing second image processing requiring a shorter processing time than the first image processing upon the radiation image.

2. The information processing device according to claim 1, wherein, upon receiving an instruction for acquiring the second radiation image, the display control unit controls the display unit such that the first radiation image is not displayed and the second radiation image is displayed.

3. The information processing device according to claim 1, wherein the display control unit controls the display unit such that the second radiation image and first image processing information representing the first image processing are displayed and controls the display unit such that the first radiation image and the first image processing information are displayed.

4. The information processing device according to claim 1, wherein the display control unit controls the display unit such that a display of the first radiation image and a display of the second radiation image are switched in response to an instruction from an operator.

5. The information processing device according to claim 1, wherein, when an instruction for acquiring the first radiation image is received and a setting for displaying the first radiation image is made, the display control unit controls the display unit such that the first radiation image is displayed first.

6. The information processing device according to claim 1, wherein the display control unit controls the display unit such that the second radiation image is displayed in a display region used for a setting of imaging information that is one of a first display region of the display unit and a second display region of the display unit different from the first display region, and controls the display unit such that the first radiation image is displayed in the other one of the first display region and the second display region.

7. The information processing device according to claim 1, wherein the first image processing is image processing using machine learning.

8. The information processing device according to claim 1, wherein the first image processing is noise reduction processing using machine learning and the second image processing is noise reduction processing not using machine learning.

9. The information processing device according to claim 1, wherein the instruction is given by an operator before acquisition of the radiation image.

10. An information processing device comprising a display control unit configured to, upon receiving an instruction for acquiring a first radiation image, display the first radiation image on a first display unit and display a second radiation image on a second display unit different from the first display unit, the first radiation image being obtained by performing first image processing upon a radiation image obtained by detecting a radiation, the second radiation image being obtained by performing second image processing requiring a shorter processing time than the first image processing upon the radiation image.

11. The information processing device according to claim 10, wherein the first display unit has a higher resolution than the second display unit.

12. A radiation imaging system comprising:
a radiation imaging device configured to detect a radiation; and
an information processing device communicably connected to the radiation imaging device, the information processing device including
a display control unit configured to, upon receiving an instruction for acquiring a first radiation image, control a display unit such that a second radiation image is displayed first and then the first radiation image is displayed, the first radiation image being obtained by performing first image processing upon a radiation image obtained by detecting a radiation, the second radiation image being obtained by performing second image processing requiring a shorter processing time than the first image processing upon the radiation image.

13. An information processing method comprising a display control step of, upon receiving an instruction for acquiring a first radiation image, controlling a display unit such that a second radiation image is displayed first and then the first radiation image is displayed, the first radiation image being obtained by performing first image processing upon a radiation image obtained by detecting a radiation, the second radiation image being obtained by performing second image processing requiring a shorter processing time than the first image processing upon the radiation image.

14. A non-transitory computer readable storage medium instructions that, when executed by a processor of an information processing device, executes a control method, the control method comprising upon receiving an instruction for acquiring a first radiation image, controlling a display unit such that a second radiation image is displayed first and then the first radiation image is displayed, the first radiation image being obtained by performing first image processing upon a radiation image obtained by detecting a radiation, the second radiation image being obtained by performing second image processing requiring a shorter processing time than the first image processing upon the radiation image.
